(19) United States
(12) Patent Application Publication
(10) Pub. No.: US 2024/0350524 A1
(43) Pub. Date: Oct. 24, 2024

(54) DOSING REGIMENS FOR USE IN PREVENTING RELAPSE OF ACUTE MYELOID LEUKEMIA FOLLOWING ALLOGENEIC HEMATOPOIETIC STEM CELL TRANSPLANTATION WITH VENETOCLAX IN COMBINATION WITH AZACITIDINE

(71) Applicant: ABBVIE INC., North Chicago, IL (US)

(72) Inventors: Ahmed H. SALEM, Libertyville, IL (US); Johannes WOLFF, Puyallup, WA (US); John HAYSLIP, Satellite Beach, FL (US)

(73) Assignee: ABBVIE INC., North Chicago, IL (US)

(21) Appl. No.: 18/627,239

(22) Filed: Apr. 4, 2024

Related U.S. Application Data
(60) Provisional application No. 63/494,739, filed on Apr. 6, 2023.

(51) Int. Cl. A61K 31/706; A61K 31/496; A61P 35/02

(52) U.S. Cl. CPC A61K 31/706; A61K 31/496; A61P 35/02

(57) ABSTRACT

The invention described herein relates to relates to dosing methods for a human subject with acute myeloid leukemia (AML) following allogeneic hematopoietic stem cell transplantation, comprising administering to a human subject venetoclax in combination with azacitidine, wherein the human subject has previously undergone allogeneic hematopoietic stem cell transplantation.

### CROSS-REFERENCE TO RELATED APPLICATIONS

This application claims the benefit of U.S. Provisional Application No. 63/494, 739, filed Apr. 6, 2023, the disclosure of which is incorporated by reference herein in its entirety.

### FIELD OF THE INVENTION

This invention relates to dosing methods for preventing relapse of acute myeloid leukemia (AML) in human subjects following allogeneic hematopoietic stem cell transplantation, comprising administering to a human subject venetoclax in combination with azacitidine, wherein the human subject has previously undergone allogeneic hematopoietic stem cell transplantation. This invention further relates to dosing methods for reducing risk of acute myeloid leukemia (AML) relapse in a human subject following allogeneic hematopoietic stem cell transplantation, comprising administering to a human subject venetoclax in combination with azacitidine, wherein the human subject has previously undergone allogeneic hematopoietic stem cell transplantation. This invention further relates to dosing methods for maintaining remission from acute myeloid leukemia (AML) in a human subject following allogeneic hematopoietic stem cell transplantation, comprising administering to a human subject venetoclax in combination with azacitidine, wherein the human subject has previously undergone allogeneic hematopoietic stem cell transplantation.

### BACKGROUND OF THE INVENTION

Acute myeloid leukemia (AML) is an aggressive malignancy and is the most common and second most common form of acute leukemia in adults and children, respectively. Allogeneic hematopoietic stem cell transplantation (alloHSCT) plays an important role in treatment for many patients with AML; however, morbidity and mortality risks remain high in both the adult and adolescent population. The most common unfavorable outcomes after alloHSCT are disease relapse and graft-versus-host-disease (GvHD). Approximately 40% of post-SCT AML patients will relapse and face a dismal prognosis with a 2-year survival of ≤20% (Tsirigotis et al. Bone Marrow Transplantation 2016, 51:1431-1438). Therefore, strategies with the potential to reduce the risk of disease relapse and/or maintain disease remission are urgently needed. Severe acute GvHD (aGvHD) occurs in 15-30% of patients transplanted using a sibling or matched unrelated donor, while 30-60% of patients experience chronic GvHD (cGvHD) which is associated with significant morbidity (Rashidi et al. Blood Adv. 2019, 3:1826-1836). Although therapies for preventing relapse are being actively studied, best supportive care (BSC) patients do not receive antileukemic therapy.

Venetoclax has demonstrated activity as a monotherapy in AML, however, the response rate is higher when given in combination with azacitidine and the duration of remission is longer. Azacitidine is a hypomethylating agent, which synergizes with venetoclax in treatment of AML, and has various regulatory approvals for treatment of myelodysplastic syndromes and AML globally (DiNardo et al. Blood 2019, 133:7-17). A safe and effective dose of venetoclax in combination with azacitidine to reduce the risk of relapse of AML and/or maintain remission from AML following allogeneic hematopoietic stem cell transplantation was determined.

### BRIEF SUMMARY OF THE INVENTION

One aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation. Another aspect pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle and wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

Another aspect pertains to a method for preventing relapse of acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation. Another aspect pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle and wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

Another aspect pertains to a method of reducing risk of acute myeloid leukemia (AML) relapse in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein 20 mg/m² azacitidine is administered, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation. Another aspect pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein 20 mg/m² azacitidine is administered, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle and wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

Another aspect pertains to a method of maintaining remission from acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

### BRIEF DESCRIPTION OF THE DRAWINGS

FIG. 1 shows the experimental design for the dose confirmation study (Part 1) and randomized open-label study (Part 2).

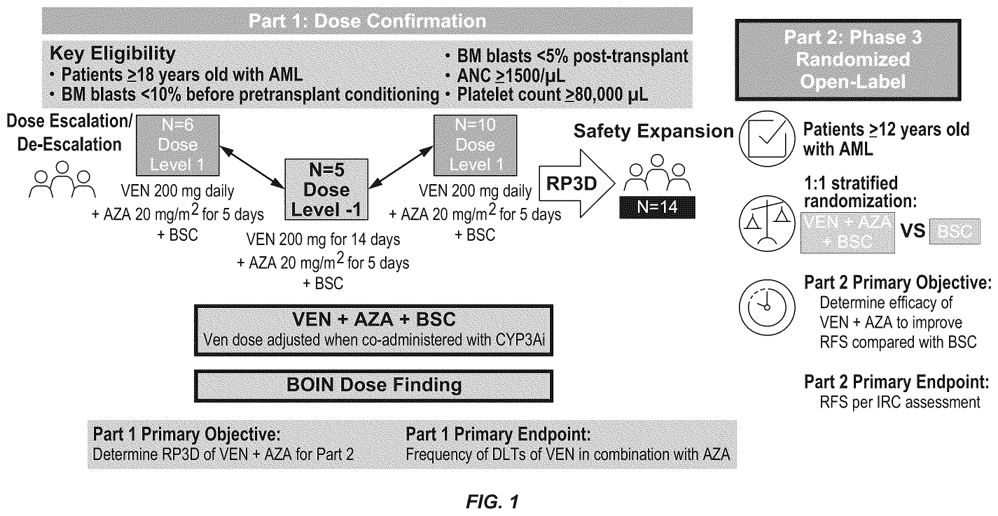

### DETAILED DESCRIPTION OF THE INVENTION

One embodiment pertains to a method for preventing relapse of acute myeloid leukemia (AML) in a human subject following allogeneic hematopoietic stem cell transplantation, comprising administering to a human subject venetoclax in combination with azacitidine, wherein the human subject has previously undergone allogeneic hematopoietic stem cell transplantation. One embodiment pertains to a method of reducing risk of acute myeloid leukemia (AML) relapse in a human subject following allogeneic hematopoietic stem cell transplantation, comprising administering to a human subject venetoclax in combination with azacitidine, wherein the human subject has previously undergone allogeneic hematopoietic stem cell transplantation. One embodiment pertains to a method of maintaining remission from acute myeloid leukemia (AML) in a human subject following allogeneic hematopoietic stem cell transplantation, comprising administering to a human subject venetoclax in combination with azacitidine, wherein the human subject has previously undergone allogeneic hematopoietic stem cell transplantation. In another embodiment, the venetoclax is administered orally. In another embodiment, the azacitidine is administered subcutaneously or intravenously. In another embodiment, the azacitidine is administered subcutaneously. In another embodiment, the azacitidine is administered intravenously. In another embodiment, 200 mg venetoclax is administered. In another embodiment, 100 mg venetoclax is administered. In another embodiment, 50 mg venetoclax is administered. In another embodiment, 30 mg venetoclax is administered. In another embodiment, 20 mg/m² azacitidine is administered.

One embodiment pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein, following the six cycles, venetoclax is orally daily administered in the absence of azacitidine for eighteen cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, and wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein, following the six cycles, venetoclax is orally daily administered in the absence of azacitidine for eighteen cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, and wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method of reducing risk of acute myeloid leukemia (AML) relapse in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, and wherein, following the six cycles, venetoclax is orally daily administered in the absence of azacitidine for eighteen cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, wherein the venetoclax is administered orally daily for a 28-day cycle, wherein the azacitidine is administered daily for the first five days of the cycle, wherein the method is practiced for six cycles, wherein, following the six cycles, venetoclax is orally daily administered in the absence of azacitidine for eighteen cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, and wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 200 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method of maintaining remission from acute myeloid leukemia (AML) in a human subject following allogeneic hematopoietic stem cell transplantation, comprising administering to a human subject 100 mg venetoclax orally per day in combination with 20 mg/m² azacitidine, wherein the venetoclax is administered for 28-days of a 28-day cycle, wherein the azacitidine is administered subcutaneously or intravenously daily for the first five days of the cycle, wherein the treatment is administered for a total of six cycles, wherein the treatment after six cycles is followed by treatment with venetoclax in the absence of azacitidine for eighteen cycles, for a total of twenty-four cycles of treatment, and wherein the human subject has previously undergone allogeneic hematopoietic stem cell transplantation.

One embodiment pertains to a method for preventing relapse of acute myeloid leukemia (AML) in a human subject following allogeneic hematopoietic stem cell transplantation, comprising administering to a human subject venetoclax orally in combination with azacitidine, wherein BSC is provided throughout treatment.

One embodiment pertains to a method of reducing risk of acute myeloid leukemia (AML) relapse in a human subject following allogeneic hematopoietic stem cell transplantation, comprising administering to a human subject venetoclax orally in combination with azacitidine, wherein BSC is provided throughout treatment.

One embodiment pertains to a method of maintaining remission from acute myeloid leukemia (AML) in a human subject following allogeneic hematopoietic stem cell transplantation, comprising administering to a human subject venetoclax orally in combination with azacitidine, wherein BSC is provided throughout treatment.

It is understood that BSC (best supportive care) is to be provided and determined for each human subject individually, based on investigator and institutional guidelines.

In certain embodiments, the method disclosed herein prevents relapse of AML in the human subject having previously undergone allogeneic hematopoietic stem cell transplantation.

In certain embodiments, the method disclosed herein reduces the risk of AML relapse in the human subject having previously undergone allogeneic hematopoietic stem cell transplantation.

In certain embodiments, the risk of AML relapse is reduced according to the methods disclosed herein.

In certain embodiments, the method disclosed herein maintains remission from AML in the human subject having previously undergone allogeneic hematopoietic stem cell transplantation.

Azacitidine is a nucleoside metabolic inhibitor with a molecular formula of C₈H₁₂N₄O₅ and a molecular weight of 244 g/mol. The chemical name is: 4-amino-1-ß-D-ribofuranosyl-striazin-2 (1H)-one and the chemical structural is:

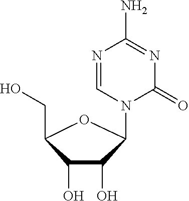

Azacitidine is a pyrimidine nucleoside analog of cytidine that inhibits DNA/RNA methyltransferases. Azacitidine is believed to exert its antineoplastic effects by causing hypomethylation of DNA and direct cytotoxicity on abnormal hematopoietic cells in the bone marrow. Hypomethylation may restore normal function to genes that are critical for differentiation and proliferation. The cytotoxic effects of azacitidine cause the death of rapidly dividing cells, including cancer cells that are no longer responsive to normal growth control mechanisms.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose. To the extent documents incorporated by reference contradict the disclosure contained in the specification; the specification will supersede any contradictory material.

So that the disclosure may be more readily understood, select terms are defined below.

### DEFINITIONS

As used in the specification and the appended claims, unless specified to the contrary, the following terms have the meaning indicated:

The term “AE” means an adverse event in a clinical trial. The AEs are graded per CTCAE (Common Terminology Criteria for Adverse Events). The CTCAE guideline provides standardized definitions for the AEs and are used widely across all oncology trials.

The term “alloHSCT” means allogeneic hematopoietic stem cell transplantation. The term “allogeneic stem cell transplantation” is understood to mean “allogeneic hematopoietic stem cell transplantation.”

The term “ALT” means alanine transaminase, a liver enzyme that is released into the bloodstream indicating damage to the liver.

The term “AML” means acute myeloid leukemia.

The term “ANC” means absolute neutrophil count, the percentage of neutrophils in a white blood count.

The term “AST” means aspartate aminotransferase, an enzyme found in the liver and muscles. Damage to liver cells releases this enzyme into the blood.

The term “AZA” means azacitidine.

The term “best supportive care” or “BSC” means the standard of care without any further treatment for AML.

The term “BM” means bone marrow.

The term “BOIN” means Bayesian Optimal Interval Design, a common tool in Phase 1 oncology trials.

The term “BSC” means best supportive care.

The term “Cockcroft Gault formula” is a means of assessing renal function through creatinine clearance.

The term “CYP3Ai” means CYP3A inhibitor.

The term “strong CYP3A inhibitor” means a drug that increases the AUC of sensitive index substrates of a given CYP3A≥5-fold.

The term “moderate CYP3A inhibitor” means a drug that increases the AUC of sensitive index substrates of a given CYP3A≥2 to <5-fold.

In embodiments, examples of strong CYP3A inhibitors include boceprevir, clarithromycin, cobicistat, conivaptan, danoprevir/ritonavir, elvitegravir/ritonavir, idelalisib, indinavir, itraconazole, ketoconazole, mibefradil, lopinavir/ritonavir, nefazodone, nelfinavir, paritaprevir/ritonavir combinations, posaconazole, ritonavir, saquinavir, telaprevir, telithromycin, tipranavir/ritonavir, troleandomycin, and voriconazole.

In embodiments, examples of moderate CYP3A inhibitors include amprenavir, aprepitant, atazanavir, cimetidine, ciprofloxacin, clotrimazole, conivaptan, crizotinib, cyclosporine, diltiazem, dronedarone, erythromycin, fluconazole, fosamprenavir, isavuconazole, fluvoxamine, imatinib, isavuconazole, tofisopam, and verapamil.

The term “DLT” means dose-limiting toxicity, which is a pre-established standard based on severity of adverse events during the trial, which results in lowering the dose of the drug substance being tested.

The term “G-CSF” means the administration of granulocyte colony-stimulating factor to combat severe neutropenia.

The term “GGT” means gamma-glutamyl transferase activity, an enzymatic activity associated with cardiovascular disease.

The term “GvHD” means graft versus host disease, a common disease following stem cell transplants.

The term “Hy's law case” defined as elevated aspartate aminotransferase (AST) or alanine aminotransferase (ALT)≥3×ULN with concurrent elevated total bilirubin≥2×ULN in the absence of elevated alkaline phosphatase or other conditions that could cause these laboratory abnormalities.

The term “N/A” means not applicable.

The term “QD” means once daily.

The terms “patient” and “subject” are used herein interchangeably.

The term “RP3D” means recommended phase three dose.

The term “RFS” means relapse free survival. The term “subject” means a human.

The terms “treat”, “treating” and “treatment” refer to a method of alleviating or abrogating a disease and/or its attendant symptoms.

The term “ULN” means upper limit of normal and is defined as the 95th percentile of the target population.

The term “VEN” means venetoclax.

The term “WHO” is the World Health Organization.

The use of the terms “a” and “an” and “the” and similar referents in the context of describing the invention (especially in the context of the following claims) are to be construed to cover both the singular and the plural, unless otherwise indicated herein or clearly contradicted by context.

### EXAMPLES

In order that the invention described herein may be more fully understood, the following example is set forth.

### Example 1

### Phase 3 Trial of Venetoclax in the VIALE-T Study of Venetoclax Plus Azacitidine as Maintenance After Allogeneic Hematopoietic Stem Cell Transplantation in Patients With AML (VIALE-T)

VIALE-T is a Phase 3, randomized, open-label trial that evaluates the safety and efficacy of venetoclax+azacitidine compared to best supportive care (BSC) as maintenance therapy in patients with AML after alloHSCT.

### Key Eligibility Criteria:

Subjects must have been ≥18 years old for Part 1 (dose confirmation+safety expansion phase) and at least 12 years old for Part 2 (randomization phase). Subjects must have been diagnosed with AML by World Health Organization Criteria (WHO; 2017) and either been planning for allogeneic hematopoietic stem cell transplantation or had received allogeneic transplantation within the past 14 days. All types and WHO categories of AML and any number of previous lines of therapy may have been included.

The key laboratory requirements were as follows:
Blast percentage in bone marrow before transplant<10%
Blast percentage in bone marrow after transplant<5%
Laboratory values meeting the following criteria prior to Cycle 1 Day 1
   Bilirubin ≤3×ULN* within 7 days prior to Cycle 1 Day 1 (*Subjects with Gilbert's Syndrome may have had a bilirubin concentration of >3×ULN per discussion between the investigator and medical director
   For Part 1: ANC≥1,500/μL, measured twice at least 48 hours apart and within one week from Cycle 1 Day 1. Subjects should not have had granulocyte-colony stimulating factor (G-CSF) treatment for 7 days before first ANC count.
   For Part 2: ANC≥1,000/μL, measured twice at least 48 hours apart and within one week from Cycle 1 Day 1. Subjects should not have had granulocyte-colony stimulating factor (G-CSF) treatment for 7 days before first ANC count.
   Part 1: Platelet count≥80,000/μL measured twice at least 48 hours apart and within 1 week from Cycle 1 Day 1. Subjects should not have had a platelet transfusion for 7 days before first platelet count.
   Part 2: Platelet count≥ 50,000/μL measured twice at least 48 hours apart and within 1 week from treatment start. Subjects should not have had a platelet transfusion for 7 days before first platelet count.
Adequate renal function as demonstrated by a creatinine clearance>30 mL/minute; calculated by the Cockcroft Gault formula or measured by 24-hour urine collection. Subjects 12-17 years old must have had the equivalent as calculated by the Schwartz formula.
Subjects ≥17 years old must have a Karnofsky Performance Scale (KPS) score >50 and subjects 12-≤16 years old must have had a Lansky Play Performance Scale score >40.

Patients ≥18 years old with AML in remission who received alloHSCT as consolidation were enrolled in three cohorts. The Bayesian Optimal Interval (BOIN) design guided dose escalation and de-escalation was based upon the cumulative number of dose-limiting toxicities (DLTs) observed at the current dose level. The first cohort of 6 patients received a dose level 1 (DL1) of 200 mg venetoclax daily for 28 days of each 28-day cycle+20 mg/m² of azacitidine daily for 5 days+BSC. A second cohort of 5 patients received a dose level-1 (DL-1) of 200 mg venetoclax daily for 14 days of each 28-day cycle+20 mg/m² of azacitidine daily for 5 days+BSC. A third cohort of 10 patients were re-tested at DL1 prior to safety expansion, which included 14 additional patients at DL1. The drug combination was administered for a total of 6 cycles, followed by venetoclax administration in the absence of azacitidine for an additional 18 cycles.

Tables 1 and 2: Baseline Demographics and Grade 3 or Higher Toxicities Observed in ≥20% Patients Treated with DL 1 and DL-1 of Venetoclax in Combination with Azacitidine During the Safety Expansion Phase (Part 1).

| | Dose | Dose | All |
|---|---|---|---|
| | Level 1 | Level −1 | Patients |
| | (N = 30) | (N = 5) | (N = 35) |
| Median age (range), years | 58.5 (20-77) | 50 (45-60) | 58 (20-77) |
| Male, n (%) | 19 (63) | 2 (40) | 21 (60) |
| Race, n (%) | | | |
| Asian | 5 (17) | 1 (20) | 6 (17) |
| White | 24 (80) | 4 (80) | 28 (80) |
| Missing | 1 (3) | 0 | 1 (3) |
| AML type, n (%) | | | |
| De novo | 25 (83) | 4 (80) | 29 (83) |
| Secondary | 5 (17) | 1 (20) | 6 (17) |
| Cytogenetic risk, n (%) | | | |
| Favorable | 1 (3) | 0 | 1 (3) |
| Intermediate | 16 (53) | 1 (20) | 17 (49) |
| Poor | 11 (37) | 2 (40) | 13 (37) |
| Unknown/missing | 2 (7) | 2 (40) | 4 (11) |
| Prior treatment with Ven, | 7 (23) | 0 | 7 (20) |
| n (%) | | | |
| Prior treatment with Aza, | 6 (20) | 0 | 6 (17) |
| n (%) | | | |
| Hepatic impairment, n (%) | | | |
| Moderate | 1 (3) | 0 | 1 (3) |
| Severe | 0 | 0 | 0 |
| Renal impairment, n (%) | | | |
| Moderate | 6 (20) | 0 | 6 (17) |
| Severe | 0 | 0 | 0 |
| Response prior to transplant, | | | |
| n (%) | | | |
| CR | 19 (63) | 4 (80) | 23 (66) |
| CR2 or later | 3 (10) | 0 | 3 (9) |
| Non-CR | 8 (27) | 1 (20) | 9 (26) |

TABLE 2

| | Dose Level 1 | Dose Level −1 | All Patients |
|---|---|---|---|
| | (N = 30) | (N = 5) | (N = 35) |
| Any TEAE, n (%) | 30 (100) | 5 (100) | 35 (100) |
| Any serious TEAE, n (%) | 8 (27) | 2 (40) | 10 (29) |
| Any TEAE leading to, n (%) | | | |
| Ven discontinuation | 6 (20) | 2 (40) | 8 (23) |
| Aza discontinuation | 7 (23) | 1 (20) | 8 (23) |
| Ven dose interruption | 20 (67) | 3 (60) | 23 (66) |
| Aza dose interruption | 16 (53) | 2 (40) | 18 (51) |
| Ven dose reduction | 12 (40) | 1 (20) | 13 (37) |
| Aza dose reduction | 6 (20) | 0 | 6 (17) |
| Any TEAE leading to death, | 2 (7) | 0 | 2 (6) |
| n (%) | | | |
| Most Common TEAEsª, | Any Grade/ | Any Grade/ | Any Grade/ |
| n (%) | Grade | Grade | Grade |
| | 3/4 | 3/4 | 3/4 |
| Neutropenia^{b} | 20 (67)/15 (50) | 3 (60)/3 (60) | 23 (66)/18 (51) |
| Thrombocytopenia^{c} | 17 (57)/10 (33) | 2 (40)/2 (40) | 19 (54)/12 (34) |
| Nausea | 15 (50)/0 | 2 (40)/0 | 17 (49)/0 |
| Diarrhea | 10 (33)/2 (7) | 1 (20)/0 | 11 (31)/2 (6) |
| Vomiting | 9 (30)/0 | 1 (20)/0 | 10 (29)/0 |
| Abdominal pain | 8 (27)/1 (3) | 0/0 | 8 (23)/1 (3) |
| Fatigue | 8 (27)/0 | 0 /1 (20) | 8 (23)/1 (3) |
| ALT increased | 7 (23)/2 (7) | 2 (40)/1 (20) | 9 (26)/3 (9) |
| Anemia | 7 (23)/5 (17) | 1 (20)/1 (20) | 8 (23)/6 (17) |
| AST increased | 7 (23)/1 (3) | 1 (20)/0 | 8 (23)/1 (3) |

ªIncludes TEAEs occurring in >20% of all patients.
^{b}Includes preferred terms of neutropenia and neutrophil count decreased.
^{c}Includes preferred terms of thrombocytopenia and platelet count decreased.

Patients were counted once in each row regardless of the number of events they may have experienced.

Antimicrobial agents and/or antifungal agents are commonly administered to patients after alloHCST to confer antimicrobial and/or antifungal prophylaxis, thereby avoiding infection. Many antimicrobial agents and/or antifungal agents administered to patients after alloHCST are known to also have CYP3A inhibition activity. However, the use of CYP3A inhibitors has previously been shown to interact with venetoclax and azacitidine therapy against AML (Jonas et al, Blood. 2020, 136 (Supplement 1): 50-52.). To counter this interaction, venetoclax dose was modified as is shown in Table 3. Following cessation of using CYP3A inhibitors, venetoclax dose was increased to full rate after 3 days.

TABLE 3
Dose Modifications for Venetoclax: Moderate
and Strong CYP3A Inhibitor Use

| | Venetoclax Dose | Venetoclax Dose |
|---|---|---|
| Assigned | if Co-Administered | if Co-Administered |
| Venetoclax | with a Moderate | with a Strong |
| Dose | CYP3A Inhibitor | CYP3A Inhibitor |
| 200 mg | 100 mg | 50 mg (30 mg, if posaconazole) |

CYP3A = cytochrome P450 3A

Venetoclax dose adjustments were not added or combined. If dosing more than one medication in combination with Venetoclax, the highest recommended dose reduction was performed.

⅘ (80%) and 13/16 (81.3%) patients enrolled in the DL-1 and DL1 groups respectively in dose escalation/de-escalation, were DLT evaluable. No DLT was observed at DL-1. At DL1, the overall DLT rate was 3/13 patients (23.1%); 2/13 patients (15.4%) experienced neutropenia (Grade ≥3 lasting ≥14 days), and 1/13 patients (7.7%) experienced Grade 2 diarrhea resulting in >20% venetoclax dose omission.

For Part 1 of the study, ⅗ (60%) and 22/30 (73.3%) patients who received DL-1 and DLI respectively, experienced a Grade 3 or 4 treatment-emergent adverse event (TEAE), the most common being neutropenia, followed by thrombocytopenia. Supportive measures such as the use of growth factors was allowed for management. Furthermore, both neutrophil and platelet counts were either stable or improved for patients who continued on the study drugs. 8/30 (26.7%) patients in the DL1 group and ⅖ (40%) patients in the DL-1 group experienced a serious adverse event (SAE), the most common being sepsis and diarrhea.

Cytopenias were among the most commonly occurring treatment-emergent adverse events and were manageable with supportive measures, as indicated by neutrophil and platelet levels over time.

Based on the review of composite safety data in Part 1 of the study, dose level 1 (200 mg Ven×28-days and Aza 20 mg/m²×5 days) was selected as the recommended Phase 3 dose (RPTD) for the Part 2 randomization phase.

As of the filing date, the status of the 30 subjects in the dose level 1 (DL1) group in Part 1 was as follows:

5 subjects completed 24 cycles of therapy without relapse. Of these 5 subjects, 4 remain on therapy after 24 cycles and 1 discontinued therapy after completing 24 cycles.

6 subjects have not relapsed, remain on therapy, but have not yet completed 24 cycles of therapy.

10 subjects have discontinued therapy prior to 24 cycles due to relapse. Of these 10 subjects, 5 experienced morphological relapse, 2 experienced clinical relapse, 2 experienced both morphological and clinical relapse, and 1 subject discontinued therapy for a reason other than relapse but then experienced morphological relapse 177 days after discontinuation of therapy.

9 subjects discontinued therapy prior to 24 cycles for reasons other than relapse. Of these 9 subjects, 3 withdrew from the study for convenience, 2 experienced cytopenia, 1 experienced multiorgan failure, 1 experienced hemodynamic deterioration, 1 experienced sepsis, and 1 withdrew due to the primary investigator's decision.

As of the filing date, the status of 5 subjects in the dose level-1 (DL-1) group in Part 1 was as follows:

1 subject completed 24 cycles of therapy without relapse. This subject discontinued therapy after completing 24 cycles.

No subjects discontinued therapy prior to 24 cycles due to relapse.

4 subjects discontinued therapy prior to 24 cycles for reasons other than relapse. Of these 4 subjects, 3 experienced cytopenia, and 1 discontinued therapy due to experiencing GvHD.

1. A method for preventing relapse of acute myeloid leukemia (AML) in a human subject following allogeneic hematopoietic stem cell transplantation, comprising administering to a human subject 200 mg venetoclax orally per day in combination with azacitidine, wherein the azacitidine is administered subcutaneously or intravenously, and wherein the human subject has previously undergone allogeneic hematopoietic stem cell transplantation.

2. The method of claim 1, wherein 20 mg/m² azacitidine is administered.

3. A method for preventing relapse of acute myeloid leukemia (AML) in a human subject following allogeneic hematopoietic stem cell transplantation, comprising administering daily to a human subject 100 mg venetoclax orally per day in combination with 20 mg/m² azacitidine, wherein the subject is concurrently undergoing treatment with a moderate CYP3A inhibitor, wherein the azacitidine is administered subcutaneously or intravenously, and wherein the human subject has previously undergone allogeneic hematopoietic stem cell transplantation.

4. A method for preventing relapse of acute myeloid leukemia (AML) in a human subject following allogeneic hematopoietic stem cell transplantation, comprising administering daily to a human subject 50 mg venetoclax orally per day in combination with 20 mg/m² azacitidine, wherein the subject is concurrently undergoing treatment with a strong CYP3A inhibitor, wherein the azacitidine is administered subcutaneously or intravenously, and wherein the human subject has previously undergone allogeneic hematopoietic stem cell transplantation.

5. A method for preventing relapse of acute myeloid leukemia (AML) in a human subject following allogeneic hematopoietic stem cell transplantation, comprising administering daily to a human subject 30 mg venetoclax orally per day in combination with 20 mg/m² azacitidine, wherein the subject is concurrently undergoing treatment with posaconazole, wherein the azacitidine is administered subcutaneously or intravenously, and wherein the human subject has previously undergone allogeneic hematopoietic stem cell transplantation.

6. The method of claim 1, wherein the venetoclax is administered orally daily for a 28-day cycle, and wherein the azacitidine is administered daily for the first five days of the cycle.

7. The method of claim 6, wherein the method is practiced for six cycles.

8. The method of claim 1, wherein the venetoclax is administered orally daily for a 28-day cycle, and wherein the azacitidine is administered daily for the first five days of the cycle, wherein the method is practiced for six cycles, and wherein, following the six cycles, venetoclax is orally daily administered in the absence of azacitidine for eighteen cycles.

9. The method of claim 1, wherein the method comprises administering the 200 mg venetoclax orally per day in combination with 20 mg/m² of the azacitidine, wherein the venetoclax is administered for 28-days of a 28-day cycle, wherein the azacitidine is administered subcutaneously or intravenously daily for the first five days of the cycle, and wherein the treatment is administered for a total of six cycles.

10. The method of claim 3, wherein the method comprises administering the 100 mg venetoclax orally per day in combination with 20 mg/m² of the azacitidine, wherein the venetoclax is administered for 28-days of a 28-day cycle, wherein the azacitidine is administered subcutaneously or intravenously daily for the first five days of the cycle, and wherein the treatment is administered for a total of six cycles, and wherein the human subject has previously undergone allogeneic hematopoietic stem cell transplantation.

11. The method of claim 1, wherein the method comprises administering the 200 mg venetoclax orally per day in combination with 20 mg/m² of the azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the treatment after six cycles is followed by treatment with venetoclax in the absence of azacitidine for eighteen cycles, for a total of twenty-four cycles of treatment.

12. The method of claim 3, wherein the method comprises administering the 100 mg venetoclax orally per day in combination with 20 mg/m² of the azacitidine, wherein the venetoclax is administered for 28-days of a 28-day cycle, wherein the azacitidine is administered subcutaneously or intravenously daily for the first five days of the cycle, wherein the treatment is administered for a total of six cycles, and wherein the treatment after six cycles is followed by treatment with venetoclax in the absence of azacitidine for eighteen cycles, for a total of twenty-four cycles of treatment.
